(19) United States
(12) Patent Application Publication
(10) Pub. No.: US 2025/0249208 A1
(43) Pub. Date: Aug. 7, 2025

(54) Optical-Fiber Stylet Holders and Methods Thereof

(71) Applicant: Bard Access Systems, Inc., Salt Lake City, UT (US)

(72) Inventors: Steffan Sowards, Salt Lake City, UT (US); Anthony K. Misener, Bountiful, UT (US); William Robert McLaughlin, Bountiful, UT (US)

(21) Appl. No.: 19/188,439

(22) Filed: Apr. 24, 2025

Related U.S. Application Data
(63) Continuation of application No. 17/529,022, filed on Nov. 17, 2021, now Pat. No. 12,285,572.
(60) Provisional application No. 63/115,442, filed on Nov. 18, 2020.

(51) Int. Cl. A61M 25/01; A61B 1/07

(52) U.S. Cl. CPC A61M 25/0102; A61B 1/07

(57) ABSTRACT

Optical-fiber stylet holders and methods for holding optical-fiber stylets in position in catheters or the like prevent breakage of optical fibers in the optical-fiber stylets and maintain functionality of the optical-fiber stylets. The holding of optical-fiber stylets in position in catheters or the like can be important for maintaining distal tips of intravascularly delivered optical-fiber stylets in their target anatomical locations during procedures.

### PRIORITY

This application is a continuation of U.S. patent application Ser. No. 17/529,022, filed Nov. 17, 2021, now U.S. Pat. No. 12,285,572, which claims the benefit of priority to U.S. Provisional Application No. 63/115,442, filed Nov. 18, 2020, each of which is incorporated by reference in its entirety into this application.

### BACKGROUND

Intravascular guidance of medical devices including guidewires, catheters, and the like have often used fluoroscopic methods for guiding distal tips of such medical devices through vasculatures and determining whether the distal tips are appropriately placed in their target anatomical locations. However, the fluoroscopic methods expose patients and their attending clinicians to harmful X-ray radiation. Moreover, the patients can be exposed to potentially harmful contrast media needed for the fluoroscopic methods. For these reasons, some current medical research has turned to developing optical methods such has fiber-optic shape-sensing (“FOSS”) methods for the intravascular guidance of medical devices.

Current FOSS methods for the intravascular guidance of medical devices utilize optical-fiber stylets having fiber Bragg grating (“FBG”) sensors along their length for shape sensing with the optical-fiber stylets. Being made of drawn glass or plastic, such optical-fiber stylets are prone to breakage when folded or “doubled over.” However, folding stylets over onto catheters from which the stylets are intravascularly delivered is a common way to hold the stylets in position once their distal tips are appropriately placed in their target anatomical locations. Indeed, FIG. 11 shows how a proximal portion of a stylet 10 extending from a Luer connector 12 is folded over an extension leg 14 of a catheter 16 such as a peripherally inserted central catheter (“PICC”) or a central venous catheter (“CVC”) to hold the stylet 10 in position. But FIG. 11 also shows how such a stylet can result in breakage if the stylet is an optical-fiber stylet. What is needed is an optical-fiber stylet holder for holding optical-fiber stylets in position in catheters or the like while maintaining functionality of the optical-fiber stylets.

Disclosed herein are optical-fiber stylet holders and methods thereof for holding optical-fiber stylets in position in catheters or the like while maintaining functionality of the optical-fiber stylets.

### SUMMARY

Disclosed herein is an optical-fiber stylet holder including, in some embodiments an integrated funnel. The integrated funnel includes a radiused shoulder and a neck. The radiused shoulder has a radius configured to allow an optical-fiber stylet to be placed against the radiused shoulder without breakage of one or more optical fibers in the optical-fiber stylet. The neck has an inner diameter sufficient to accept the optical-fiber stylet, and the neck is configured to be coincident with a lumen of an extension leg of a catheter.

In some embodiments, the optical-fiber stylet holder further includes a locking mechanism. The locking mechanism is configured to lock the optical-fiber stylet in the optical-fiber stylet holder.

In some embodiments, the locking mechanism includes a plurality of notches about a mouth of the integrated funnel through a side wall of the optical-fiber stylet holder. Each notch of the plurality of notches is sized to firmly hold the optical-fiber stylet when the optical-fiber stylet is pressed therein.

In some embodiments, the locking mechanism includes a plurality of side-wall petals about the mouth of the integrated funnel alternating with the plurality of notches. Each side-wall petal of the plurality of side-wall petals is configured to direct the optical-fiber stylet into an adjacent notch when the optical-fiber stylet is pressed thereagainst.

In some embodiments, the locking mechanism includes a cap configured to cover both a mouth of the integrated funnel and the optical-fiber stylet when extending from the mouth. The cap is coupled to a side wall of the optical-fiber stylet holder by a living hinge.

In some embodiments, the optical-fiber stylet holder is integral with a Luer connector of the catheter.

In some embodiments, the optical-fiber stylet holder is a cap including internal threads in a distal portion of the cap configured to screw onto external threads of a Luer connector of the catheter.

Also disclosed herein is an optical-fiber stylet holder including, in some embodiments, a cap, a cylindrical gasket, and a locking mechanism. The cap includes internal threads and a cap through hole. The internal threads are in a distal portion of a bore of the cap, and the internal threads are configured to screw onto complementary external threads of an insertable piece. The cap through hole is in a proximal end of the cap having an inner diameter sufficient to accept an optical-fiber stylet. The cylindrical gasket is of a compressible material. The gasket includes a gasket through hole having an inner diameter no smaller than that of the cap through hole. The locking mechanism is defined by the gasket disposed in the cap between the proximal end of the cap and the insertable piece. The locking mechanism is configured to lock the optical-fiber stylet in the optical-fiber stylet holder by compression of the gasket when the cap is screwed onto the insertable piece with the optical-fiber stylet in the optical-fiber stylet holder.

In some embodiments, the compression of the gasket is axial compression between the proximal end of the cap and the insertable piece. The axial compression, in turn, provides radial compression around the optical-fiber stylet to lock the optical-fiber stylet in the optical-fiber stylet holder.

In some embodiments, the insertable piece is part of a male Luer connector of a catheter.

In some embodiments, the insertable piece is part of the optical-fiber stylet holder. The insertable piece has a distal portion configured as a female Luer connector.

Also disclosed herein is an optical-fiber stylet holder including, in some embodiments, a pair of arms, a tortuous path for an optical-fiber stylet to follow between the pair of arms, and a locking mechanism. The pair of arms is biased toward a centerline of the optical-fiber stylet holder. The locking mechanism is defined by the torturous path. The locking mechanism is configured to lock the optical-fiber stylet in the optical-fiber stylet holder by friction through a combination of the pair of arms being biased toward the centerline of the optical-fiber stylet holder and the tortuous path formed between the pair of arms.

In some embodiments, the optical-fiber stylet further includes a plurality of rollers mounted on the pair of arms. Consecutive rollers of the plurality of rollers alternate from arm to arm of the pair of arms forming a tortuous path for an optical-fiber stylet to follow.

In some embodiments, the optical-fiber stylet holder is integral with a Luer connector of the catheter.

In some embodiments, the optical-fiber stylet holder is a cap including internal threads in a distal portion of the cap configured to screw onto external threads of a Luer connector of the catheter.

Also disclosed herein is an optical-fiber stylet holder including, in some embodiments, a clamp, a through hole through a distal end of the optical-fiber stylet holder coincident with a lumen of an extension leg of a catheter, and a locking mechanism defined by the clamp. The clamp includes a first jaw, a second jaw, and an elastic piece configured to store mechanical energy. The first jaw and the second jaw longitudinally extend in a same direction as that of a centerline of the optical-fiber stylet holder. The elastic piece is coupled to both the first jaw and the second jaw, and the elastic piece is configured to hold the second jaw against the first jaw. The locking mechanism is configured to lock the optical-fiber stylet in the optical-fiber stylet holder by friction of the clamp along a length of the optical-fiber stylet.

In some embodiments, the optical-fiber stylet holder is integral with a Luer connector of the catheter.

In some embodiments, the optical-fiber stylet holder is a cap including internal threads in a distal portion of the cap configured to screw onto external threads of a Luer connector of the catheter.

Also disclosed herein is an optical-fiber stylet holder including, in some embodiments, a pair of hinge plates, a hinge formed between the pair of hinge plates, and an elastomeric pad disposed on a hinge plate of the pair of hinge plates. Each hinge plate of the pair of hinge plates includes a fastener complementary to the other. The hinge formed between the pair of hinge plates utilizes an optical-fiber stylet to provide a hinge pin of the hinge. The elastomeric pad is configured to compress the optical-fiber stylet against another hinge plate of the pair of hinge plates when the pair of hinge plates are closed. The optical-fiber stylet holder thereby provides a locking mechanism configured to lock the optical-fiber stylet in the optical-fiber stylet holder and prevent distal movement of the optical-fiber stylet while the optical-fiber stylet is disposed in a catheter.

In some embodiments, the optical-fiber stylet holder is configured to couple to a Luer connector of the catheter.

Also disclosed herein is an optical-fiber stylet holder including, in some embodiments, a pair of arms, a pair of opposing through holes, a connecting portion between the pair of arms, and a pair of opposing elastomeric pads. The pair of arms includes a stationary arm and a moveable arm. Each arm of the pair of arms includes a fastener complementary to the other. Each through hole of the pair of through holes is through at least an arm of the pair of arms. An elastomeric pad of the pair of elastomeric pads is disposed on the moveable arm and another elastomeric pad of the pair of elastomeric pads is disposed on the connecting portion. The pair of elastomeric pads are configured to compress an optical-fiber stylet inserted through the through holes when the moveable arm is moved toward the connecting portion. The optical-fiber stylet holder thereby provides a locking mechanism configured to lock the optical-fiber stylet in the optical-fiber stylet holder and prevent distal movement of the optical-fiber stylet while the optical-fiber stylet is disposed in a catheter.

In some embodiments, the stationary arm includes a rack of teeth and the moveable arm includes a pawl. The pawl is configured to move linearly across the rack of teeth as the moveable arm is moved toward the connecting portion.

In some embodiments, the optical-fiber stylet holder is configured to couple to a Luer connector of the catheter.

Also disclosed herein is an optical-fiber stylet holder including, in some embodiments, a housing, an insert, and a spring. The housing includes a housing through hole dimensioned to accept an optical-fiber stylet therethrough. The insert is partially disposed in the housing and partially extends as a button from a side of the housing perpendicular to the housing through hole. The insert includes an insert through hole coincident with the housing through hole. The spring is configured to push or pull the insert toward the side of the housing from which the insert extends. The optical-fiber stylet acts as a stop to the insert being pushed or pulled toward the side of the housing when the optical-fiber stylet is inserted through both the housing through hole and the insert through hole. The optical-fiber stylet holder thereby provides a locking mechanism configured to lock the optical-fiber stylet in the optical-fiber stylet holder and prevent distal movement of the optical-fiber stylet while the optical-fiber stylet is disposed in a catheter.

In some embodiments, the optical-fiber stylet holder is configured to couple to a Luer connector of the catheter.

Also disclosed herein is an optical-fiber stylet holder including, in some embodiments, a substrate and a torturous channel in a side of the substrate. The torturous channel is dimensioned to accept an optical-fiber stylet laid therein. The optical-fiber stylet holder thereby provides a locking mechanism configured to lock the optical-fiber stylet in the optical-fiber stylet holder and prevent distal movement of the optical-fiber stylet while the optical-fiber stylet is disposed in a catheter.

In some embodiments, the optical-fiber stylet holder is configured to couple to a Luer connector of the catheter.

Also disclosed herein is an optical-fiber stylet holder including, in some embodiments, a septum configured to be disposed in a Luer connector of a catheter. The septum includes a through hole dimensioned to accept an optical-fiber stylet therethrough and firmly hold the optical-fiber stylet by friction when the optical-fiber stylet is inserted therein. The optical-fiber stylet holder thereby provides a locking mechanism configured to lock the optical-fiber stylet in the optical-fiber stylet holder and prevent both proximal and distal movement of the optical-fiber stylet while the optical-fiber stylet is disposed in a catheter.

In some embodiments, the optical-fiber stylet holder further includes a retainer. The retainer includes an annular rim and a plurality of wedge-shaped pieces extending from the rim and biased toward a center of the retainer. The retainer is configured to slide off the Luer connector and onto the optical-fiber stylet bringing tips of the wedge-shaped pieces inward toward the center of the retainer.

In some embodiments, the retainer provides a secondary locking mechanism configured to prevent distal movement of the optical-fiber stylet while the optical-fiber stylet is disposed in a catheter.

Also disclosed herein is a method of an optical-fiber stylet holder including, in some embodiments, a stylet-inserting step, a first stylet-advancing step, a second stylet-advancing step, and a stylet-holding step. The stylet-inserting step includes inserting an optical-fiber stylet into the optical-fiber stylet holder. The first stylet-advancing step includes advancing the optical-fiber stylet through a catheter. The second stylet-advancing step includes advancing a distal tip of the optical-fiber stylet through a vasculature to a target anatomical location in a patient. The stylet-holding step includes holding the distal tip of the optical-fiber stylet in the target anatomical location without at least distal advancement of the distal tip by locking the optical-fiber stylet in the optical-fiber stylet holder.

In some embodiments, the method further includes a stylet-placing step, a first stylet-pressing step, and a second stylet-pressing step. The stylet-placing step includes placing the optical-fiber stylet against a radiused shoulder of an integrated funnel of the optical-fiber stylet holder. The first stylet-pressing step includes pressing the optical-fiber stylet against a side-wall petal of a plurality of side-wall petals about a mouth of the integrated funnel to direct the optical-fiber stylet into an adjacent notch of a plurality of notches about the mouth of the integrated funnel. The second stylet-pressing step includes pressing the optical-fiber stylet into a notch of the plurality of notches, thereby locking the optical-fiber stylet in the optical-fiber stylet holder and holding the distal tip of the optical-fiber stylet in the target anatomical location.

In some embodiments, the method further includes the stylet-placing step and a mouth-covering step. Again, the stylet-placing step includes placing the optical-fiber stylet against a radiused shoulder of an integrated funnel of the optical-fiber stylet holder. The mouth-covering step includes covering a mouth of the integrated funnel with a cap coupled to a side wall of the optical-fiber stylet holder by a living hinge, thereby locking the optical-fiber stylet in the optical-fiber stylet holder and holding the distal tip of the optical-fiber stylet in the target anatomical location.

In some embodiments, the method further includes a cap-screwing step and a compressing step. The cap-screwing step includes screwing a cap of the optical-fiber stylet holder onto an insertable piece. The compressing step includes compressing around the optical-fiber stylet a cylindrical gasket disposed in the cap between a proximal end of the cap and the insertable piece with the screwing of the cap onto the insertable piece, thereby locking the optical-fiber stylet in the optical-fiber stylet holder and holding the distal tip of the optical-fiber stylet in the target anatomical location.

In some embodiments, the stylet-inserting step includes following a torturous path with the optical fiber stylet through a plurality of rollers mounted on a pair of arms biased toward a centerline of the optical-fiber stylet holder, thereby locking the optical-fiber stylet in the optical-fiber stylet holder and holding the distal tip of the optical-fiber stylet in the target anatomical location.

In some embodiments, the method further includes a clamp-opening step and a clamp-closing step. The clamp-opening step includes opening a clamp of the optical-fiber stylet holder before the stylet-inserting step. The clamp-closing step includes closing a second jaw of the clamp onto a first jaw of the clamp with the optical-fiber stylet between the second jaw and the first jaw, thereby locking the optical-fiber stylet in the optical-fiber stylet holder and holding the distal tip of the optical-fiber stylet in the target anatomical location.

In some embodiments, the method further includes a hinge-closing step. The hinge-closing step includes closing a pair of hinge plates of the optical-fiber stylet holder to compress the optical-fiber stylet with an elastomeric pad of a hinge plate of the pair of hinge plates against another hinge plate of the pair of hinge plates, thereby locking the optical-fiber stylet in the optical-fiber stylet holder and holding the distal tip of the optical-fiber stylet in the target anatomical location.

In some embodiments, the stylet-inserting step includes inserting the optical-fiber stylet into a pair of opposing through holes through a pair of arms including a stationary arm and a moveable arm.

In some embodiments, the method further includes a pawl-moving step. The paw!-moving step includes moving a pawl of the moveable arm against a rack of teeth of the stationary arm to compress the optical-fiber stylet between an elastomeric pad disposed on the moveable arm and another elastomeric pad disposed on a connecting portion of the optical-fiber stylet holder between the pair of arms, thereby locking the optical-fiber stylet in the optical-fiber stylet holder and holding the distal tip of the optical-fiber stylet in the target anatomical location.

In some embodiments, the method further includes a button-pressing step and a button-releasing step. The button-pressing step includes pressing a button of an insert extending from a side of a housing of the optical-fiber stylet holder to pull or push a spring to allow the optical-fiber stylet to freely move through both housing and insert through holes for adjusting a position of the optical-fiber stylet holder on the optical-fiber stylet. The button-releasing step includes releasing the button to push or pull the insert by way of the spring toward the side of the housing from which the insert extends, thereby locking the optical-fiber stylet in the optical-fiber stylet holder as a stop in the housing and insert through holes and holding the distal tip of the optical-fiber stylet in the target anatomical location.

In some embodiments, the stylet-inserting step includes laying the optical-fiber stylet into a torturous channel in a side of a substrate of the optical-fiber stylet holder, thereby locking the optical-fiber stylet in the optical-fiber stylet holder and holding the distal tip of the optical-fiber stylet in the target anatomical location.

In some embodiments, the stylet-inserting step includes inserting the optical-fiber stylet into a through hole of a septum of the optical-fiber stylet holder, thereby locking the optical-fiber stylet in the optical-fiber stylet holder by friction and holding the distal tip of the optical-fiber stylet in the target anatomical location.

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1 illustrates a first optical-fiber stylet holder in accordance with some embodiments.

FIG. 2 illustrates a second optical-fiber stylet holder in accordance with some embodiments.

FIG. 3 illustrates a third optical-fiber stylet holder in accordance with some embodiments.

FIG. 4 illustrates a fourth optical-fiber stylet holder in accordance with some embodiments.

FIG. 5 illustrates a fifth optical-fiber stylet holder in accordance with some embodiments.

FIG. 6A illustrates a sixth optical-fiber stylet holder in accordance with some embodiments.

FIG. 6B illustrates the sixth optical-fiber stylet holder in use with a catheter in accordance with some embodiments.

FIG. 7A illustrates a seventh optical-fiber stylet holder in accordance with some embodiments.

FIG. 7B illustrates the seventh optical-fiber stylet holder in use with catheter in accordance with some embodiments.

FIG. 8 illustrates an eighth optical-fiber stylet holder in accordance with some embodiments.

FIG. 9 illustrates a ninth optical-fiber stylet holder in accordance with some embodiments.

FIG. 10 illustrates a tenth optical-fiber stylet holder in accordance with some embodiments.

FIG. 11 illustrates a common way to hold stylets in position once their distal tips are appropriately placed in their target anatomical locations, which way is unsuitable for optical-fiber stylets.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

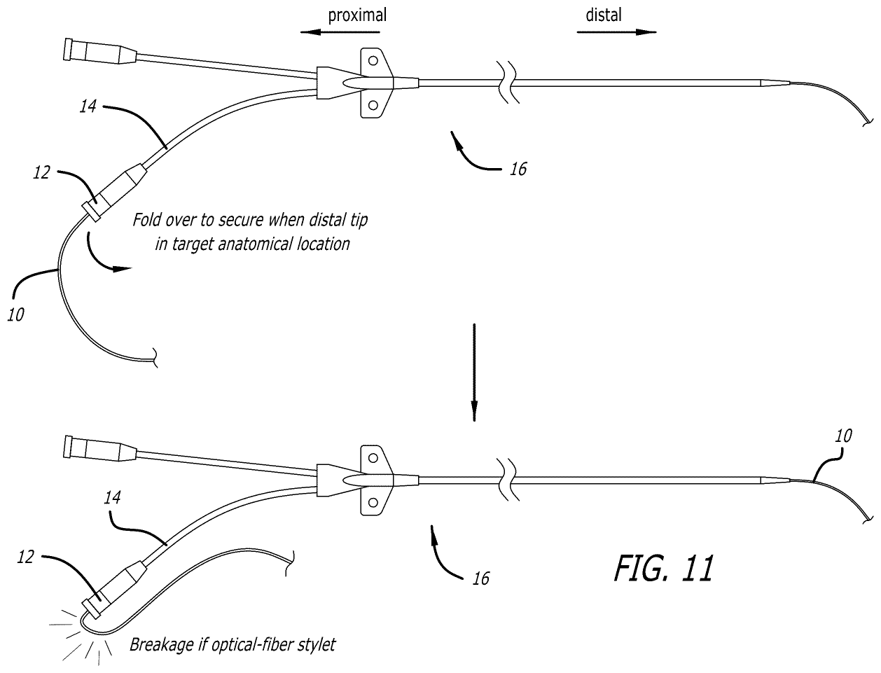

As set forth above, folding stylets over onto catheters from which the stylets are intravascularly delivered is a common way to hold the stylets in position once their distal tips are appropriately placed in their target anatomical locations. Indeed, FIG. 11 shows how the proximal portion of the stylet 10 extending from the Luer connector 12 is folded over the extension leg 14 of the catheter 16 such as a PICC or a CVC to hold the stylet 10 in position. But FIG. 11 also shows how such a stylet can result in breakage if the stylet is an optical-fiber stylet drawn from glass or plastic. What is needed is an optical-fiber stylet holder for holding optical-fiber stylets in position in catheters or the like while maintaining functionality of the optical-fiber stylets. Disclosed herein are optical-fiber stylet holders and methods thereof for holding optical-fiber stylets in position in catheters or the like while maintaining functionality of the optical-fiber stylets.

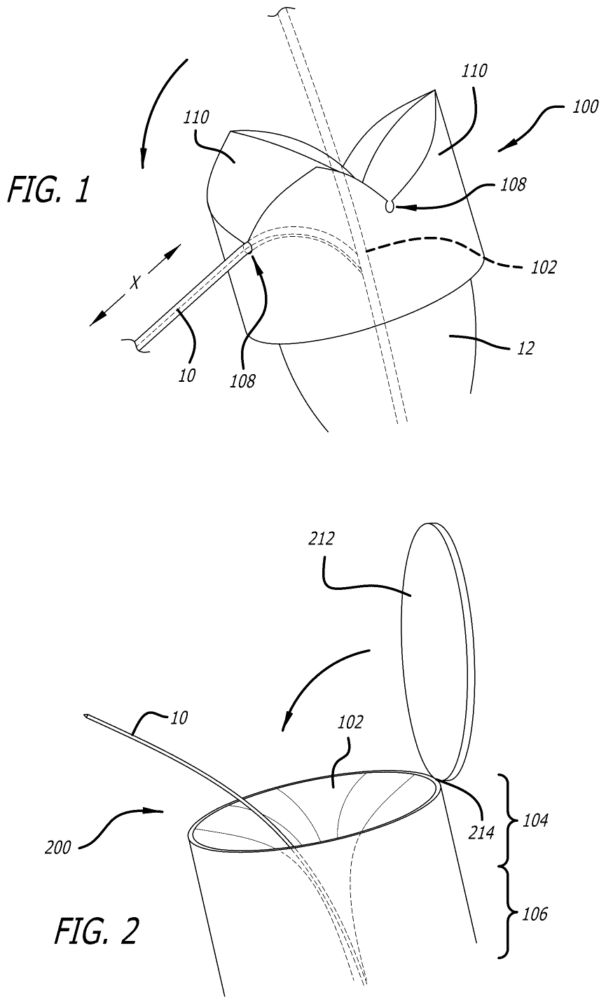

FIGS. 1 and 2 respectively illustrate a first optical-fiber stylet holder 100 and a second optical-fiber stylet holder 200 in accordance with some embodiments.

As shown, the optical-fiber stylet holder 100 or 200 includes an integrated funnel 102 and a locking mechanism configured to lock an optical-fiber stylet such as the optical-fiber stylet 10 in the optical-fiber stylet holder 100 or 200, thereby preventing proximal or distal movement of the optical-fiber stylet 10.

The integrated funnel 102 includes a radiused shoulder 104 and a neck 106. The radiused shoulder 104 has a radius configured to allow the optical-fiber stylet 10 to be placed against the radiused shoulder 104 without breakage of one or more optical fibers in the optical-fiber stylet 10. Advantageously, the radius of the radiused shoulder 104, being known, can be used for FOSS system calibration. The neck 106 has an inner diameter sufficient to accept the optical-fiber stylet 10. The neck is configured to be coincident with a lumen of an extension leg of a catheter such as the extension leg 14 of the catheter 16 for advancing the optical-fiber stylet 10 through the catheter 16. The integrated funnel 102 can be formed of a semi-flexible material.

As shown in FIG. 1, the locking mechanism can include a plurality of notches 108 about a mouth of the integrated funnel 102 through a side wall of the optical-fiber stylet holder 100. Each notch of the plurality of notches 108 is sized to firmly hold the optical-fiber stylet 10 when the optical-fiber stylet 10 is pressed therein. In addition, the locking mechanism can include a plurality of side-wall petals 110 about the mouth of the integrated funnel 102 alternating with the plurality of notches 108. Each side-wall petal of the plurality of side-wall petals 110 is configured to direct the optical-fiber stylet 10 into an adjacent notch when the optical-fiber stylet is pressed thereagainst.

As shown in FIG. 2, the locking mechanism includes a cap 212 configured to cover both a mouth of the integrated funnel 102 and the optical-fiber stylet 10 when the optical-fiber stylet 10 extends from the mouth. The cap 212 is coupled to a side wall of the optical-fiber stylet holder 200 by a living hinge 214.

The optical-fiber stylet holder 100 or 200 can be integral with the Luer connector 12 of the catheter 16. Alternatively, the optical-fiber stylet holder 100 or 200 is a cap including internal threads in a distal portion of the cap configured to screw onto external threads of the Luer connector 12 of the catheter 16. Since Luer connectors are standardized, the latter embodiment is advantageous in that the optical-fiber stylet holder 100 or 200 can be used with existing PICCs, CVC, or the like.

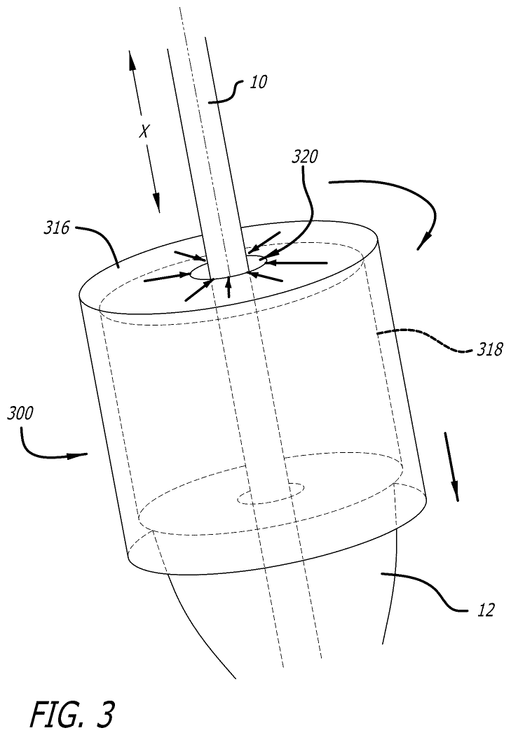

FIG. 3 illustrates a third optical-fiber stylet holder 300 in accordance with some embodiments.

As shown, the optical-fiber stylet holder 300 includes a cap 316, a cylindrical gasket 318, and a locking mechanism configured to lock an optical-fiber stylet such as the optical-fiber stylet 10 in the optical-fiber stylet holder 300, thereby preventing proximal or distal movement of the optical-fiber stylet 10.

The cap includes internal threads (not shown) and a cap through hole 320. The internal threads are in a distal portion of a bore of the cap 316. The internal threads are configured to screw onto complementary external threads of an insertable piece. The cap through hole 320 is in a proximal end of the cap 316. The cap through hole 320 has an inner diameter sufficient to accept the optical-fiber stylet 10 inserted therein.

The gasket 318 is of a compressible material such as an elastomeric material. The gasket 318 includes a gasket through hole (not shown) having an inner diameter no smaller than that of the cap through hole 320. The gasket through hole is configured to be coincident with a lumen of an extension leg of a catheter such as the extension leg 14 of the catheter 16 for advancing the optical-fiber stylet 10 through the catheter 16.

The locking mechanism is defined by the gasket 318 disposed in the cap 316 between the proximal end of the cap 316 and the insertable piece. The locking mechanism is configured to lock the optical-fiber stylet 10 in the optical-fiber stylet holder 300 by compression of the gasket 318 when the cap 316 is screwed onto the insertable piece with the optical-fiber stylet 10 in the optical-fiber stylet holder 300. The compression of the gasket 318 is axial compression between the proximal end of the cap 316 and the insertable piece. The axial compression, in turn, provides radial compression around the optical-fiber stylet 10 to lock the optical-fiber stylet 10 in the optical-fiber stylet holder 300.

The insertable piece can be a part (e.g., a proximal portion) of the Luer connector 12 of the catheter 16, which Luer connector 12 is a male Luer connector having external threads. A Iternatively, the insertable piece is part of the optical-fiber stylet holder 300. In the latter embodiment, the insertable piece can have a distal portion configured as a female Luer connector having internal threads complementary to the external threads of the Luer connector 12 and a proximal portion with the external threads complementary to the internal threads of the cap 316. The insertable piece as part of the optical-fiber stylet holder 300 is advantageous in that the thread pitch does not have to match that specified for Luer connectors, which are not necessarily designed for finer-tuned movements such as those beneficial for compressing the optical-fiber stylet 10 in the optical-fiber stylet holder 300.

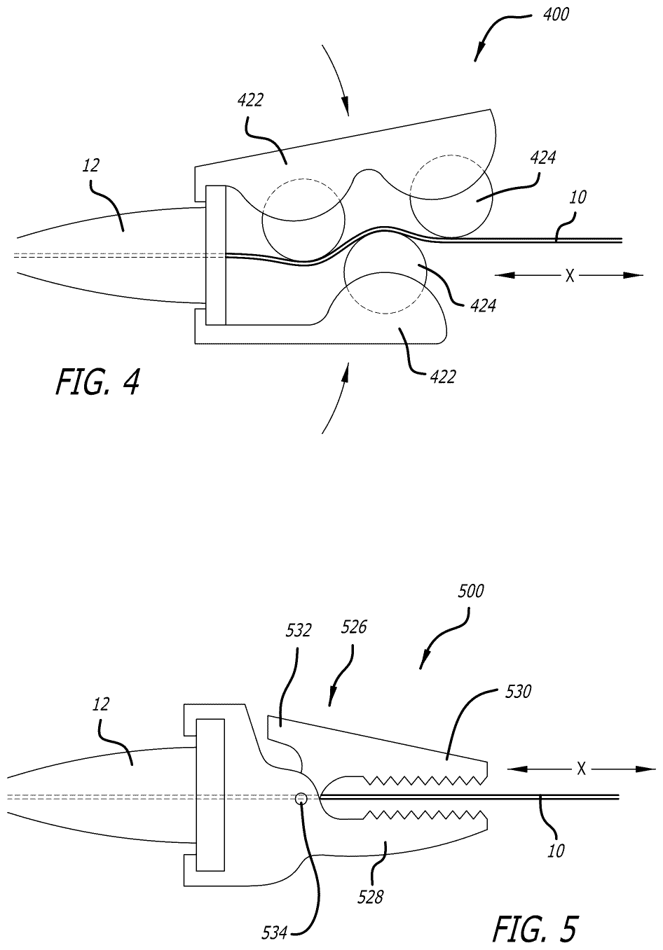

FIG. 4 illustrates a fourth optical-fiber stylet holder 400 in accordance with some embodiments.

As shown, the optical-fiber stylet holder 400 includes a pair of arms 422, a plurality of rollers 424 mounted on the pair of arms 422, and a locking mechanism configured to lock an optical-fiber stylet such as the optical-fiber stylet 10 in the optical-fiber stylet holder 400, thereby preventing proximal or distal movement of the optical-fiber stylet 10.

The pair of arms 422 is biased toward a centerline of the optical-fiber stylet holder 400. The plurality of rollers 424 is mounted on the pair of arms 422. Consecutive rollers of the plurality of rollers 424 alternate from arm to arm of the pair of arms 422 forming a tortuous path for the optical-fiber stylet 10 to follow. Advantageously, the torturous path, being known, can be used for FOSS system calibration.

The optical-fiber stylet holder 400 also includes a distal through hole distal of the plurality of rollers 424 and through a distal end of the optical-fiber stylet holder 400, which through hole is coincident with a lumen of an extension leg of a catheter such as the extension leg 14 of the catheter 16 for advancing the optical-fiber stylet 10 through the catheter 16.

The locking mechanism is defined by the plurality of rollers 424 mounted on the pair of arms 422. The locking mechanism is configured to lock the optical-fiber stylet 10 in the optical-fiber stylet holder 400 by friction through a combination of the pair of arms 422 being biased toward the centerline of the optical-fiber stylet holder 400 and the tortuous path formed by the plurality of rollers 424.

The optical-fiber stylet holder 400 can be integral with the Luer connector 12 of the catheter 16. Alternatively, the optical-fiber stylet holder 400 is a cap including internal threads in a distal portion of the cap configured to screw onto external threads of the Luer connector 12 of the catheter 16. Since Luer connectors are standardized, the latter embodiment is advantageous in that the optical-fiber stylet holder 400 can be used with existing PICCs, CVC, or the like.

FIG. 5 illustrates a fifth optical-fiber stylet holder 500 in accordance with some embodiments.

As shown, the optical-fiber stylet holder 500 includes a clamp 526, a through hole through a distal end of the optical-fiber stylet holder 500 coincident with a lumen of an extension leg of a catheter such as the extension leg 14 of the catheter 16, and a locking mechanism defined by the clamp 526 configured to lock an optical-fiber stylet such as the optical-fiber stylet 10 in the optical-fiber stylet holder 500, thereby preventing proximal or distal movement of the optical-fiber stylet 10.

The clamp includes a first jaw 528 such as a stationary jaw, a second jaw 530 such as a moveable jaw, and an elastic piece (e.g., a metal spring, a molded plastic piece having a bias toward each side of the molded plastic piece, etc.) configured to store mechanical energy (not shown). The first jaw 528 and the second jaw 530 longitudinally extend in a same direction as that of a centerline of the optical-fiber stylet holder 500. The elastic piece is coupled to both the first jaw 528 and the second jaw 530, and the elastic piece is configured to hold the second jaw 530 against the first jaw 528 or vice versa. Indeed, both the first jaw 528 and the second jaw 530 can be moveable from a common anchor point of the optical-fiber stylet holder 500. A thumb pad 532 is configured to allow a clinician to press into the clamp 526 with his or her thumb on an opposite side of a hinge pin 534 to overcome a elastic-piece force of the elastic piece holding the second jaw 530 against the first jaw 528, thereby allowing adjustment (e.g., proximal or distal movement) of the optical fiber stylet 10.

The locking mechanism is configured to lock the optical-fiber stylet 10 in the optical-fiber stylet holder 500 by friction of the clamp 526 along a length of the optical-fiber stylet 10.

The optical-fiber stylet holder 500 can be integral with the Luer connector 12 of the catheter 16. Alternatively, the optical-fiber stylet holder 500 is a cap including internal threads in a distal portion of the cap configured to screw onto external threads of the Luer connector 12 of the catheter 16. Since Luer connectors are standardized, the latter embodiment is advantageous in that the optical-fiber stylet holder 500 can be used with existing PICCs, CVC, or the like.

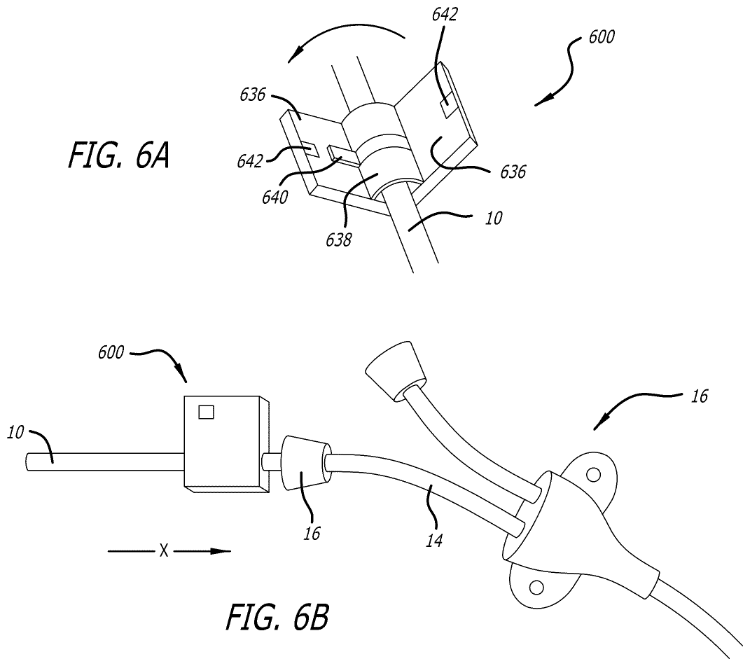

FIG. 6A illustrates a sixth optical-fiber stylet holder 600 in accordance with some embodiments. FIG. 6B illustrates the sixth optical-fiber stylet holder 600 in use with a catheter in accordance with some embodiments.

As shown, the optical-fiber stylet holder 600 includes a pair of hinge plates 636, a hinge 638 formed between the pair of hinge plates 636, and an elastomeric pad 640 disposed on a hinge plate of the pair of hinge plates 636.

Each hinge plate of the pair of hinge plates 636 includes a fastener complementary to the other making a pair of fasteners 642.

The hinge 638 formed between the pair of hinge plates 636 utilizes an optical-fiber stylet such as the optical-fiber stylet 10 to provide a hinge pin of the hinge 638.

The elastomeric pad 640 is configured to compress the optical-fiber stylet 10 against another hinge plate of the pair of hinge plates 636 when the pair of hinge plates 636 are closed.

The optical-fiber stylet holder 600 thereby provides a locking mechanism configured to lock the optical-fiber stylet 10 in the optical-fiber stylet holder 600 and prevent distal movement of the optical-fiber stylet 10 while the optical-fiber stylet 10 is disposed in a catheter such as the catheter 16. However, the optical-fiber stylet holder 600 can be configured to couple to a Luer connector of the catheter 16 to prevent proximal movement of the optical-fiber stylet 10 as well.

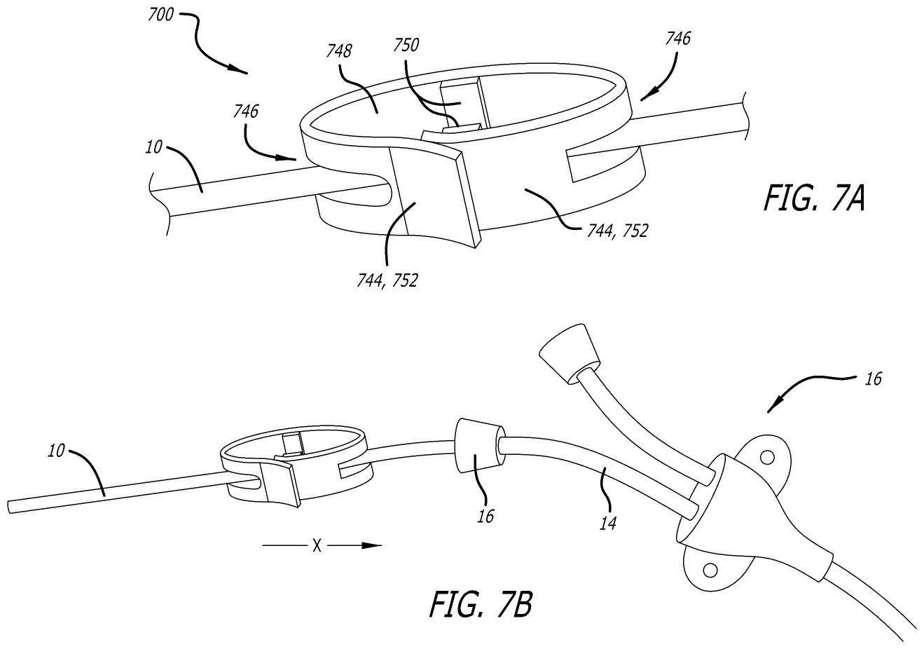

FIG. 7A illustrates a seventh optical-fiber stylet holder 700 in accordance with some embodiments. FIG. 7B illustrates the seventh optical-fiber stylet holder 700 in use with catheter in accordance with some embodiments.

As shown, the optical-fiber stylet holder 700 includes a pair of arms 744, a pair of opposing through holes 746, a connecting portion 748 between the pair of arms 744, and a pair of opposing elastomeric pads 750.

The pair of arms 744 includes a stationary arm 752 and a moveable arm 754. Each arm of the pair of arms 744 includes a fastener complementary to the other. For example, the stationary arm 752 can include a rack of teeth and the moveable arm 754 can include a pawl. When present, the pawl is configured to move linearly across the rack of teeth as the moveable arm 754 is moved toward the connecting portion 748 of the optical-fiber stylet holder 700.

Each through hole of the pair of through holes 746 is through at least an arm of the pair of arms 744.

An elastomeric pad of the pair of elastomeric pads 750 is disposed on the moveable 754 arm and another elastomeric pad of the pair of elastomeric pads 750 is disposed on the connecting portion 748 of the optical-fiber stylet holder 700. The pair of elastomeric pads 750 are configured to compress an optical-fiber stylet such as the optical-fiber stylet 10 inserted through the pair of through holes 746 when the moveable arm 754 is moved toward the connecting portion 748 of the optical-fiber stylet holder 700.

The optical-fiber stylet holder 700 thereby provides a locking mechanism configured to lock the optical-fiber stylet 10 in the optical-fiber stylet holder 700 and prevent distal movement of the optical-fiber stylet 10 while the optical-fiber stylet 10 is disposed in a catheter such as the catheter 16. However, the optical-fiber stylet holder 700 can be configured to couple to a Luer connector of the catheter 16 to prevent proximal movement of the optical-fiber stylet 10 as well.

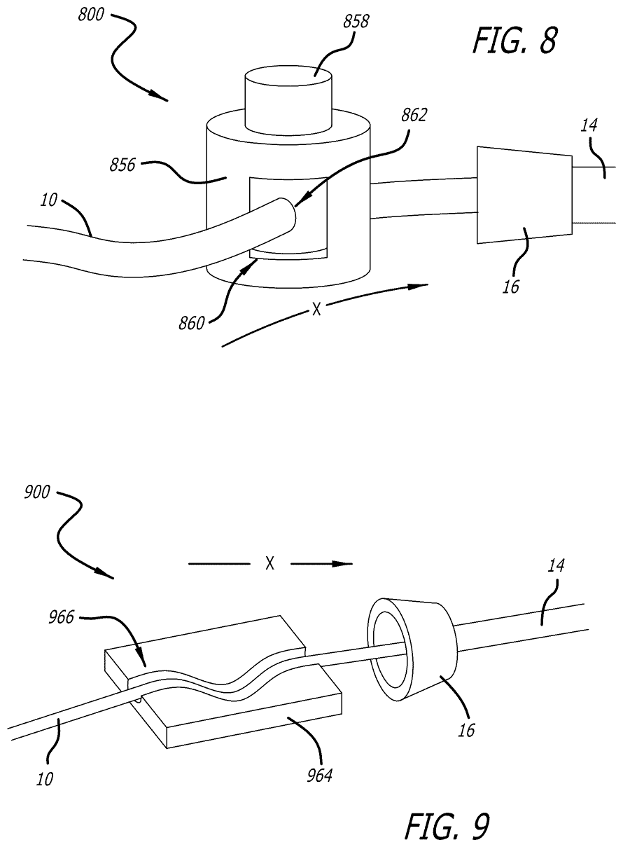

FIG. 8 illustrates an eighth optical-fiber stylet holder 800 in accordance with some embodiments.

As shown, the optical-fiber stylet holder 800 includes a housing 856, an insert 858, and a spring (not shown).

The housing 856 includes a housing through hole 860 dimensioned to accept an optical-fiber stylet such as the optical-fiber stylet 10 therethrough.

The insert 858 is partially disposed in the housing 856 and partially extends as a button from a side of the housing 856 perpendicular to the housing through hole 860. The insert includes an insert through hole 862 coincident with the housing through hole 860.

The spring is configured to push or pull the insert 858 toward the side of the housing 856 from which the insert 858 extends. The optical-fiber stylet 10 acts as a stop to the insert 858 being pushed or pulled toward the side of the housing 856 when the optical-fiber stylet 10 is inserted through both the housing through hole 860 and the insert through hole 862.

The optical-fiber stylet holder 800 thereby provides a locking mechanism configured to lock the optical-fiber stylet 10 in the optical-fiber stylet holder and prevent distal movement of the optical-fiber stylet 10 while the optical-fiber stylet 10 is disposed in a catheter such as the catheter 16. However, the optical-fiber stylet holder 800 can be configured to couple to a Luer connector of the catheter 16 to prevent proximal movement of the optical-fiber stylet 10 as well.

FIG. 9 illustrates a ninth optical-fiber stylet holder 900 in accordance with some embodiments.

As shown, the optical-fiber stylet holder 900 includes a substrate 964 and a torturous channel 966 in a side (e.g., a major side) of the substrate 964.

The torturous channel 966 is dimensioned to accept an optical-fiber stylet such as the optical-fiber stylet 10 laid therein. Advantageously, the torturous channel 966, being known, can be used for FOSS system calibration.

The optical-fiber stylet holder 900 thereby provides a locking mechanism configured to lock the optical-fiber stylet 10 in the optical-fiber stylet holder 900 and prevent distal movement of the optical-fiber stylet 10 while the optical-fiber stylet 10 is disposed in a catheter such as the catheter 16. However, the optical-fiber stylet holder 900 can be configured to couple to a Luer connector of the catheter 16 to prevent proximal movement of the optical-fiber stylet 10 as well.

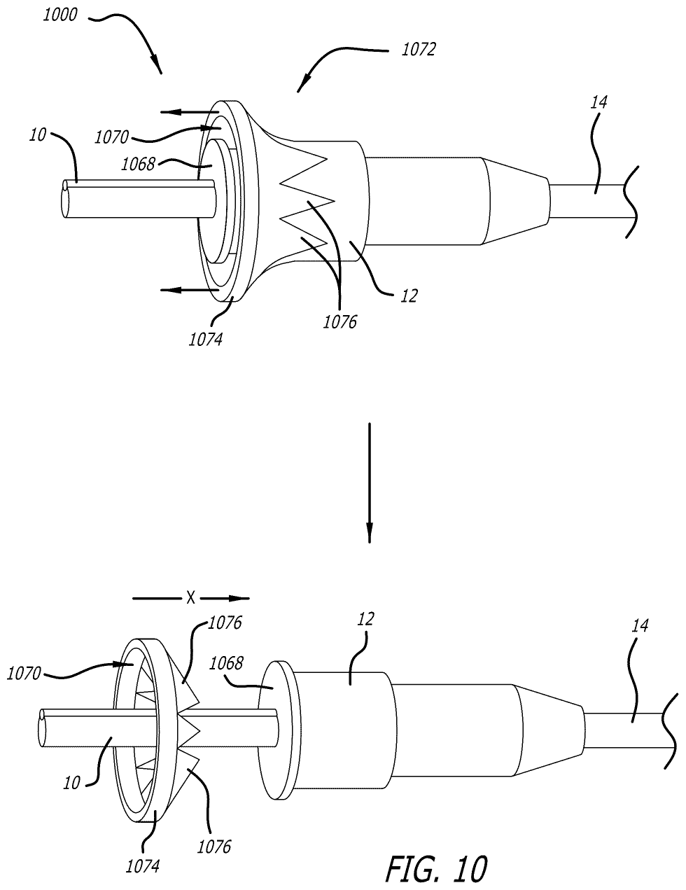

FIG. 10 illustrates a tenth optical-fiber stylet 1000 holder in accordance with some embodiments.

As shown, the optical-fiber stylet holder includes a septum 1068 configured to be disposed in a Luer connector of a catheter such as the Luer connector 12 of the catheter 16.

The septum 1068 includes a through hole 1070 dimensioned to accept an optical-fiber stylet such as the optical-fiber stylet 10 therethrough and firmly hold the optical- fiber stylet 10 by friction when the optical-fiber stylet 10 is inserted therein. The septum 1068 can be formed of an elastomeric material.

The optical-fiber stylet holder 1000 can also include a retainer 1072. The retainer 1072 includes an annular rim 1074 and a plurality of wedge-shaped pieces 1076 extending from the rim 1074 and biased toward a center of the retainer 1072. The retainer 1072 is configured to slide off the Luer connector 12 and onto the optical-fiber stylet 10 bringing tips of the wedge-shaped pieces 1076 inward toward the center of the retainer 1072.

The retainer 1072 provides a secondary locking mechanism configured to prevent distal movement of the optical-fiber stylet 10 while the optical-fiber stylet 10 is disposed in a catheter such as the catheter 16. Indeed, on account of the wedge-shaped pieces 1076 of the retainer 1072 being lodged against the optical-fiber stylet 10, the optical-fiber stylet 10 is prevented from distal movement.

The optical-fiber stylet holder thereby provides a primary locking mechanism in the septum 1070 that is configured to lock the optical-fiber stylet 10 in the optical-fiber stylet holder 1000 and prevent both proximal and distal movement of the optical-fiber stylet 10 while the optical-fiber stylet 10 is disposed in the catheter 16. The optical-fiber stylet holder also thereby provides a secondary locking mechanism configured in the retainer 1072 that is configured to lock the optical-fiber stylet 10 in the optical-fiber stylet holder 900 and prevent distal movement of the optical-fiber stylet 10 while the optical-fiber stylet 10 is disposed in the catheter 16.

Methods

Methods include methods of using the optical-fiber stylet holders 100-1000 disclosed herein. For example, a method of an optical-fiber stylet holder 100, 200, 300, 400, 500, 600, 700, 800, 900, or 1000 includes a stylet-inserting step, a first stylet-advancing step, a second stylet-advancing step, and a stylet-holding step.

The stylet-inserting step includes inserting an optical-fiber stylet such as the optical-fiber stylet 10 into the optical-fiber stylet holder 100, 200, 300, 400, 500, 600, 700, 800, 900, or 1000. The first stylet-advancing step includes advancing the optical-fiber stylet 10 through a catheter such as the catheter 16. The second stylet-advancing step includes advancing a distal tip of the optical-fiber stylet 10 through a vasculature to a target anatomical location in a patient. The stylet-holding step includes holding the distal tip of the optical-fiber stylet 10 in the target anatomical location without at least distal advancement of the distal tip by locking the optical-fiber stylet in the optical-fiber stylet holder 100, 200, 300, 400, 500, 600, 700, 800, 900, or 1000.

Again, FIG. 1 illustrates the first optical-fiber stylet holder 100 in accordance with some embodiments.

The method can further include a stylet-placing step, a first stylet-pressing step, and a second stylet-pressing step.

The stylet-placing step includes placing the optical-fiber stylet 10 against the radiused shoulder 104 of the integrated funnel 102 of the optical-fiber stylet holder 100.

The first stylet-pressing step includes pressing the optical-fiber stylet 10 against a side-wall petal of the plurality of side-wall petals 110 about the mouth of the integrated funnel 102 to direct the optical-fiber stylet 10 into an adjacent notch of the plurality of notches 108 about the mouth of the integrated funnel 102.

The second stylet-pressing step includes pressing the optical-fiber stylet 10 into a notch of the plurality of notches 108, thereby locking the optical-fiber stylet 10 in the optical-fiber stylet holder 100 and holding the distal tip of the optical-fiber stylet 10 in the target anatomical location.

A gain, FIG. 2 illustrates the second optical-fiber stylet holder 200 in accordance with some embodiments.

The method can further include the stylet-placing step and a mouth-covering step.

A gain, the stylet-placing step includes placing the optical-fiber stylet 10 against the radiused shoulder 104 of the integrated funnel 102 of the optical-fiber stylet holder 200.

The mouth-covering step includes covering the mouth of the integrated funnel 102 with the cap 212 coupled to the side wall of the optical-fiber stylet holder 200 by the living hinge 214, thereby locking the optical-fiber stylet 10 in the optical-fiber stylet holder 200 and holding the distal tip of the optical-fiber stylet 10 in the target anatomical location.

Again, FIG. 3 illustrates the third optical-fiber stylet holder 300 in accordance with some embodiments.

The method can further include a cap-screwing step and a compressing step.

The cap-screwing step includes screwing the cap 316 of the optical-fiber stylet holder 300 onto the insertable piece.

The compressing step includes compressing around the optical-fiber stylet 10 the cylindrical gasket 318 disposed in the cap 316 between a proximal end of the cap 316 and the insertable piece with the screwing of the cap 316 onto the insertable piece, thereby locking the optical-fiber stylet 10 in the optical-fiber stylet holder 300 and holding the distal tip of the optical-fiber stylet 10 in the target anatomical location.

A gain, FIG. 4 illustrates the fourth optical-fiber stylet holder 400 in accordance with some embodiments.

The stylet-inserting step can further include following the torturous path with the optical fiber stylet 10 through the plurality of rollers 424 mounted on the pair of arms 422 biased toward the centerline of the optical-fiber stylet holder 400, thereby locking the optical-fiber stylet 10 in the optical-fiber stylet holder 400 and holding the distal tip of the optical-fiber stylet 10 in the target anatomical location.

A gain, FIG. 5 illustrates the fifth optical-fiber stylet holder 500 in accordance with some embodiments.

The method can further include a clamp-opening step and a clamp-closing step.

The clamp-opening step includes opening the clamp 526 of the optical-fiber stylet holder 500 before the stylet-inserting step.

The clamp-closing step includes closing the second jaw 530 of the clamp 526 onto the first jaw 528 of the clamp 526 with the optical-fiber stylet 10 between the second jaw 530 and the first jaw 528, thereby locking the optical-fiber stylet 10 in the optical-fiber stylet holder 500 and holding the distal tip of the optical-fiber stylet 10 in the target anatomical location.

Again, FIG. 6A illustrates the sixth optical-fiber stylet holder 600 in accordance with some embodiments.

The method can further include a hinge-closing step.

The hinge-closing step includes closing the pair of hinge plates 636 of the optical-fiber stylet holder 600 to compress the optical-fiber stylet 10 with the elastomeric pad 640 of a hinge plate of the pair of hinge plates 636 against another hinge plate of the pair of hinge plates 636, thereby locking the optical-fiber stylet 10 in the optical-fiber stylet holder 600 and holding the distal tip of the optical-fiber stylet 10 in the target anatomical location.

Again, FIG. 7A illustrates the seventh optical-fiber stylet holder 700 in accordance with some embodiments.

The stylet-inserting step can further include inserting the optical-fiber stylet 10 into the pair of opposing through holes 746 through the pair of arms 744 including the stationary arm 752 and the moveable arm 754.

The method can further include a pawl-moving step.

The pawl-moving step includes moving the pawl of the moveable arm 754 against the rack of teeth of the stationary arm 752 to compress the optical-fiber stylet 10 between the elastomeric pad disposed on the moveable arm 754 and the other elastomeric pad disposed on the connecting portion 748 of the optical-fiber stylet holder 700 between the pair of arms 744, thereby locking the optical-fiber stylet 10 in the optical-fiber stylet holder 700 and holding the distal tip of the optical-fiber stylet 10 in the target anatomical location.

A gain, FIG. 8 illustrates the eighth optical-fiber stylet holder 800 in accordance with some embodiments.

The method can further include a button-pressing step and a button-releasing step.

The button-pressing step includes pressing the button of the insert 858 extending from the side of the housing 856 of the optical-fiber stylet holder 800 to pull or push the spring to allow the optical-fiber stylet 10 to freely move through both housing and insert through holes 860 and 862 for adjusting a position of the optical-fiber stylet holder 800 on the optical-fiber stylet 10.

The button-releasing step includes releasing the button to push or pull the insert 858 by way of the spring toward the side of the housing 856 from which the insert 858 extends, thereby locking the optical-fiber stylet 10 in the optical-fiber stylet holder 800 as a stop in the housing and insert through holes 860 and 862 and holding the distal tip of the optical-fiber stylet 10 in the target anatomical location.

Again, FIG. 9 illustrates the ninth optical-fiber stylet holder 900 in accordance with some embodiments.

The stylet-inserting step can further include laying the optical-fiber stylet 10 into the torturous channel 966 in the side of the substrate 964 of the optical-fiber stylet holder 900, thereby locking the optical-fiber stylet 10 in the optical-fiber stylet holder 900 and holding the distal tip of the optical-fiber stylet 10 in the target anatomical location.

Again, FIG. 10 illustrates the tenth optical-fiber stylet holder 1000 in accordance with some embodiments.

The stylet-inserting step can further include inserting the optical-fiber stylet 10 into the through hole 1070 of the septum 1068 of the optical-fiber stylet holder 1000, thereby locking the optical-fiber stylet 10 in the optical-fiber stylet holder 1000 by friction and holding the distal tip of the optical-fiber stylet 10 in the target anatomical location.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. An optical-fiber stylet holder, comprising:
a pair of arms including a stationary arm and a moveable arm, each arm of the pair of arms including a fastener complementary to the other;
a pair of opposing through holes, each through hole of the pair of through holes through at least an arm of the pair of arms;
a connecting portion between the pair of arms; and
a pair of opposing elastomeric pads, an elastomeric pad of the pair of elastomeric pads disposed on the moveable arm and another elastomeric pad of the pair of elastomeric pads disposed on the connecting portion configured to compress an optical-fiber stylet inserted through the through holes when the moveable arm is moved toward the connecting portion, the optical-fiber stylet holder thereby providing a locking mechanism configured to lock the optical-fiber stylet in the optical-fiber stylet holder and prevent distal movement of the optical-fiber stylet while the optical-fiber stylet is disposed in a catheter.

2. The optical-fiber stylet holder of claim 1, wherein the stationary arm includes a rack of teeth and the moveable arm includes a pawl configured to move linearly across the rack of teeth as the moveable arm is moved toward the connecting portion.

3. The optical-fiber stylet holder of claim 1, wherein the optical-fiber stylet holder is configured to couple to a Luer connector of the catheter.

4. An optical-fiber stylet holder, comprising:
a housing including a housing through hole dimensioned to accept an optical-fiber stylet therethrough;
an insert partially disposed in the housing and partially extending as a button from a side of the housing perpendicular to the housing through hole, the insert including an insert through hole coincident with the housing through hole; and
a spring configured to push or pull the insert toward the side of the housing from which the insert extends, the optical-fiber stylet acting as a stop to the insert being pushed or pulled toward the side of the housing when the optical-fiber stylet is inserted through both the housing through hole and the insert through hole, the optical-fiber stylet holder thereby providing a locking mechanism configured to lock the optical-fiber stylet in the optical-fiber stylet holder and prevent distal movement of the optical-fiber stylet while the optical-fiber stylet is disposed in a catheter.

5. The optical-fiber stylet holder of claim 4, wherein the optical-fiber stylet holder is configured to couple to a Luer connector of the catheter.

6. An optical-fiber stylet holder, comprising:
a substrate; and
a torturous channel in a side of the substrate dimensioned to accept an optical-fiber stylet laid therein, the optical-fiber stylet holder thereby providing a locking mechanism configured to lock the optical-fiber stylet in the optical-fiber stylet holder and prevent distal movement of the optical-fiber stylet while the optical-fiber stylet is disposed in a catheter.

7. The optical-fiber stylet holder of claim 6, wherein the optical-fiber stylet holder is configured to couple to a Luer connector of the catheter.

8. An optical-fiber stylet holder, comprising:
a septum configured to be disposed in a Luer connector of a catheter, the septum including a through hole dimensioned to accept an optical-fiber stylet therethrough and firmly hold the optical-fiber stylet by friction when the optical-fiber stylet is inserted therein, thereby providing a locking mechanism configured to lock the optical-fiber stylet in the optical-fiber stylet holder and prevent both proximal and distal movement of the optical-fiber stylet while the optical-fiber stylet is disposed in a catheter.

9. The optical-fiber stylet holder of claim 8, further comprising a retainer including an annular rim and a plurality of wedge-shaped pieces extending from the rim and biased toward a center of the retainer, the retainer configured to slide off the Luer connector and onto the optical-fiber stylet bringing tips of the wedge-shaped pieces inward toward the center of the retainer.

10. The optical-fiber stylet holder of claim 9, wherein the retainer provides a secondary locking mechanism configured to prevent distal movement of the optical-fiber stylet while the optical-fiber stylet is disposed in a catheter.

11. A method of an optical-fiber stylet holder, comprising:
inserting an optical-fiber stylet into the optical-fiber stylet holder;
advancing the optical-fiber stylet through a catheter;
advancing a distal tip of the optical-fiber stylet through a vasculature to a target anatomical location in a patient; and
holding the distal tip of the optical-fiber stylet in the target anatomical location without at least distal advancement of the distal tip by locking the optical-fiber stylet in the optical-fiber stylet holder.

12. The method of claim 11, further comprising:
placing the optical-fiber stylet against a radiused shoulder of an integrated funnel of the optical-fiber stylet holder;
pressing the optical-fiber stylet against a side-wall petal of a plurality of side-wall petals about a mouth of the integrated funnel to direct the optical-fiber stylet into an adjacent notch of a plurality of notches about the mouth of the integrated funnel; and
pressing the optical-fiber stylet into a notch of the plurality of notches, thereby locking the optical-fiber stylet in the optical-fiber stylet holder and holding the distal tip of the optical-fiber stylet in the target anatomical location.

13. The method of claim 11, further comprising:
placing the optical-fiber stylet against a radiused shoulder of an integrated funnel of the optical-fiber stylet holder; and
covering a mouth of the integrated funnel with a cap coupled to a side wall of the optical-fiber stylet holder by a living hinge, thereby locking the optical-fiber stylet in the optical-fiber stylet holder and holding the distal tip of the optical-fiber stylet in the target anatomical location.

14. The method of claim 11, further comprising:
screwing a cap of the optical-fiber stylet holder onto an insertable piece; and
compressing around the optical-fiber stylet a cylindrical gasket disposed in the cap between a proximal end of the cap and the insertable piece with the screwing of the cap onto the insertable piece, thereby locking the optical-fiber stylet in the optical-fiber stylet holder and holding the distal tip of the optical-fiber stylet in the target anatomical location.

15. The method of claim 11, wherein the inserting of the optical-fiber stylet into the optical-fiber stylet holder includes following a torturous path with the optical fiber stylet through a plurality of rollers mounted on a pair of arms biased toward a centerline of the optical-fiber stylet holder, thereby locking the optical-fiber stylet in the optical-fiber stylet holder and holding the distal tip of the optical-fiber stylet in the target anatomical location.

16. The method of claim 11, further comprising:
opening a clamp of the optical-fiber stylet holder before the inserting of the optical-fiber stylet into the optical-fiber stylet holder; and
closing a second jaw of the clamp onto a first jaw of the clamp with the optical-fiber stylet between the second jaw and the first jaw, thereby locking the optical-fiber stylet in the optical-fiber stylet holder and holding the distal tip of the optical-fiber stylet in the target anatomical location.

17. The method of claim 11, further comprising closing a pair of hinge plates of the optical-fiber stylet holder to compress the optical-fiber stylet with an elastomeric pad of a hinge plate of the pair of hinge plates against another hinge plate of the pair of hinge plates, thereby locking the optical-fiber stylet in the optical-fiber stylet holder and holding the distal tip of the optical-fiber stylet in the target anatomical location.

18. The method of claim 11, wherein the inserting of the optical-fiber stylet into the optical-fiber stylet holder includes inserting the optical-fiber stylet into a pair of opposing through holes through a pair of arms including a stationary arm and a moveable arm.

19. The method of claim 18, further comprising moving a pawl of the moveable arm against a rack of teeth of the stationary arm to compress the optical-fiber stylet between an elastomeric pad disposed on the moveable arm and another elastomeric pad disposed on a connecting portion of the optical-fiber stylet holder between the pair of arms, thereby locking the optical-fiber stylet in the optical-fiber stylet holder and holding the distal tip of the optical-fiber stylet in the target anatomical location.

20. The method of claim 11, further comprising:
pressing a button of an insert extending from a side of a housing of the optical-fiber stylet holder to pull or push a spring to allow the optical-fiber stylet to freely move through both housing and insert through holes for adjusting a position of the optical-fiber stylet holder on the optical-fiber stylet; and
releasing the button to push or pull the insert by way of the spring toward the side of the housing from which the insert extends, thereby locking the optical-fiber stylet in the optical-fiber stylet holder as a stop in the housing and insert through holes and holding the distal tip of the optical-fiber stylet in the target anatomical location.

21. The method of claim 11, wherein the inserting of the optical-fiber stylet into the optical-fiber stylet holder includes laying the optical-fiber stylet into a torturous channel in a side of a substrate of the optical-fiber stylet holder, thereby locking the optical-fiber stylet in the optical-fiber stylet holder and holding the distal tip of the optical-fiber stylet in the target anatomical location.

22. The method of claim 11, wherein the inserting of the optical-fiber stylet into the optical-fiber stylet holder includes inserting the optical-fiber stylet into a through hole of a septum of the optical-fiber stylet holder, thereby locking the optical-fiber stylet in the optical-fiber stylet holder by friction and holding the distal tip of the optical-fiber stylet in the target anatomical location.
